(19) United States
(12) Patent Application Publication
(10) Pub. No.: US 2006/0239907 A1
(43) Pub. Date: Oct. 26, 2006

(54) Stealthy nano agents

(75) Inventors: David Luzzi, Walllingford, PA (US); Brian W. Smith, Philadelphia, PA (US)

Correspondence Address: WOODCOCK WASHBURN LLP, ONE LIBERTY PLACE, 46TH FLOOR, 1650 MARKET STREET, PHILADELPHIA, PA, 19103, US

(73) Assignee: The Trustees of the University of Pennsylvania, Philadelphia, PA (US)

(21) Appl. No.: 10/559,445

(22) PCT Filed: Jun. 3, 2004
(86) PCT No.: PCT/US04/17396

§ 371 Date: May 9, 2006

Related U.S. Application Data
(60) Provisional application No. 60/475,526, filed on Jun. 3, 2003.
(60) Provisional application No. 60/495,369, filed on Aug. 15, 2003.

(51) Int. Cl. A61K 51/00

(52) U.S. Cl. 424/1.11; 424/1.49; 977/906

(57) ABSTRACT

Stealthy nanoagents are provided comprising inorganic shells containing pluralities of nanoagents. The nanoagents are isolated from the environment of the shells. Thereapeutics, imaging and diagnostic methods are also provided.

### CROSS REFERENCE TO RELATED APPLICATIONS

This application claims priority to U.S. Provisional Application Ser. No. 60/495,369 filed Aug. 15, 2003 and U.S. Provisional Application Ser. No. 60/475,526 filed Jun. 3, 2003, each of which is incorporated by reference herein in its entirety.

### FIELD OF THE INVENTION

The present invention is directed to nanoagents for therapeutics, diagnostics, imaging and other medical and animal health uses. The invention is also directed to scientific and industrial uses such as in sensors, polymer systems, nano-scale systems and for other things. Methods of making.

Certain references illustrate aspects of the background of this invention. See: Rossetti, R.; Brus L.; Electron-Hole Recombination Emission as a Probe of Surface Chemistry in Aqueous CdS Colloids, J. Phys. Chem., 22, 172 (1982); A. R. Kortan, R. Hull, R. L. Opila, M. G. Bawendi, M. L. Steigerwald, P. J. Carroll, and L. E. Brus; Nucleation and Growth of CdSe on ZnS Quantum Crystallite Seeds, and Vice Versa, in Inverse Micelle Media, J. Am. Chem. SOC., 112, 1327-1332 (1990); Murray C., Norris D., Bawendi M.; Synthesis and Characterization of Nearly Monodisperse CdE (E=S, Se, Te) Semiconductor Nanocrystallites, J. Am. Chem. Soc., 115, (1993); Hines M., Guyot-Sionnest P.; Synthesis and Characterization of Strongly Luminescent ZnS-Capped CdSe Nanocrystals, J. Phys. Chem. August 1995; and A. L. Rogach, L. Katsikas, A. Kornowski, D. Su, A. Eychmüller, H. Weller. Ber. Bunsenges; Water Soluble CdTe, Phys. Chem. 100, 1772-1714 (1996).

### SUMMARY OF THE INVENTION

Nano scale materials for imaging, diagnostics and therapeutics in medicine and in animal health science have begun to become important. Additionally, nano scale materials now find use in many scientific and industrial applications. In prior applications, however, nano scale agents—“nanoagents”—have been in contact with their environment. While, in some cases, this is a desirable circumstance, a number of situations have now been found to exist and more will be discovered where physical contact of nanoagent with an environment in which they are placed is not desirable. The present invention provides “stealthy” nanoagents—nanoagents largely or completely isolated from their proximate environment by the interposition of another material.

This invention features “stealthy” nanoagents, nanoscale objects useful themselves for various purposes, which are partially or wholly isolated from an environment by being encased within inorganic shells. The inorganic shells are, themselves, preferably on the nano scale, such as from about 5 to about 500 nanometers in at least one dimension. In this way, the operation of the nanoagents may take place largely or completely free from the influence of the environment surrounding the shells. The resulting, hybrid, materials offer important advantages for application in sensors, diagnostic devices and therapeutic devices. In certain manifestations, the hybrid materials could also be components of therapies, or be the therapeutic agent themselves. Hybrid materials in accordance with the invention are comprised of inorganic shell having a lumen in which the lumen contains nanoparticles, material clusters, or certain types of functional molecules. The shape of the hybrid materials will be dictated by the shape of the inorganic shell. It is not necessary that the interior species fill all of the space within the lumen of the inorganic shell for the functioning of the hybrid material.

### BRIEF DESCRIPTION OF THE DRAWINGS

FIG. 1 is a depiction of an inorganic shell with access from the lumen of the shell to the outside of the shell.

FIG. 2 shows the shell of FIG. 1 substantially filled with nanoagents.

FIG. 3 is a cross section of the shell of FIG. 1 showing a coating of inorganic material enclosing the shell.

FIG. 4 is also a cross section of the shell of FIG. 1. It is coated with an organic coating to which ligands, L are associated or attached.

FIG. 5 shows a partial cross section of a carbon nanotube with lumen and coating to which ligands are attached.

In accordance with certain preferred embodiments, compositions are provided comprising a plurality of inorganic, preferably biocompatible shells having inner spaces or lumens. It is preferred that at least one dimension of the shells be on the order of from about 5 to about 500 nanometers in size as measured by any convenient measuring methodology. The lumens of the shells contain a plurality of nanoagents. Such nanoagents are nano scale objects having a function useful in the situs for which the compositions are intended. For most biological applications, especially imaging, diagnostics and therapeutics, the shells are associated with one or more targeting ligands. Such ligands, which are known to the biological arts per se, are selected to be specifically bindable or associatable with a preselected biological target. The function of the ligands is to cause the filled shells to associate with or “stick to” particular biological structures or tissues such that the contents of the shells—the nanoagents—will perform their function in such proximity. At the same time, at least most of the nanoagents will not be in contact with the biological environment, the same being isolated within the shells.

One example of targeting ligand useful in many biological systems is the family of antibodies. Antibodies are know to “carry” objects “to” preselected sites in a biological system by virtue of the well-known and characterized immunogenic reactions appertaining to such antibodies. Similarly, attachment of antibodies to objects is also generally well understood such that attachment of antibodies to filled shells as contemplated hereby will be readily attainable by persons of ordinary skill in the art. By judicious selection of antibodies to serve as targeting ligands, direction of filled shells to desired locales in biological systems may be attained. Other targeting ligands are also known, such as the family of proteins, many of which engage in specific interactions with biological targets. One example of this is the protein transferring which, when employed as a targeting ligand, causes objects to which it is attached to localize preferentially in the vicinity of enzymes operating upon transferring. Other systems are also known as are other forms of targeting ligands. While one ligand may be attached to any given shell, pluralities of ligands per shell are preferred to improve targeting efficiency. This plurality of ligands could be composed of multiple copies of the same ligand per shell, or multiple differently targeting ligands per shell.

For many preferred embodiments, it is desired to coat shells filled with nanoagents with one or more coatings. Coatings may serve to seal the nanoagents within the shells and can also facilitate attachment or association of targeting ligand to the shells. The coatings are generally biocompatible and may be applied in single or multiple layers. Exemplary coatings include lipid layers, such as phospholipids, polymers, such as polyalkylene polyols, especially polyethylene glycol, and other species. In some embodiments, multiple layers of phospholipids is preferred. It will be appreciated that bonding targeting ligands, such as antibodies, to such coatings is known and may be employed here.

The shells useful in the practice of this invention are inorganic. Such materials include a wide variety of ceramics, glasses and other inorganic species, so long as they have internal voids or lumens. Hollow ceramic bodies, such as spheres of magnesia or alumina, are preferred for some embodiments, although other ceramics and glasses may be used as may bodies having internal porosity rather than an intact, singular lumen. The shells, which may be of any shape, must be capable of being filled with nanoagents, of holding the nanoagents and of delivering them into biological, scientific or industrial loci substantially intact and in a way that the nanoagents are largely or completely isolated from the environment external to the shells. In addition to ceramic, glass and similar materials, shells may also be comprised of SP² bonded carbon atoms—e.g. in a Fullerene arrangement. Such structures, known as carbon nanotubes (or other fullerenes having lumens) may be employed to good effect in this invention. Fullerenes may be either unilamellar or multilamellar or may comprise cage structures, all of which are known to persons of skill in the art.

It will be understood that the nanoagents filling the shells will be functional. Thus, they will give rise to some property, action or signal at a predictable time and under predictable conditions, which activity, property or signal is of use either in therapeutics, imaging, diagnosis, scientific inquiry, or industrial procedure. Additionally, the shells may be associated themselves with one or more properties such as fluorescence, phosphorescence, or radiodensity to provide further functionality for the hybrid materials of this invention. The nanoagents may comprise quantum dots, known per se, which may be used for imaging in several known ways. For example, quantum dots associated with antibodies are commercially available for imaging of biological structures through the dots' emission of radiation at a known frequency when irradiated. Other quantum dot properties may also be used beneficially in the practice of one or more embodiments of this invention. Nanoagents may also comprise nanoparticles having radionuclides. Such nanoagents provide therapeutic or imaging radiation for medical purposes. As these are essentially isolated from biological tissue, control of radiation dosage and proximity may be had. Also, relatively large doses of radiation may be attained with a relatively small number of targeting ligands as large nanoagent particles can populate the lumen of shells.

Other nanoagents may comprise dense atoms to provide radiolucency or marking. Dyes of various sorts, markers, reporter molecules, such as molecules which respond to specific types of radiation in predictable and detectable ways may also be employed. In some contexts, organic molecules comprising many dyes and markers may be seen by some not to be nano scale objects at all. Within the context of this invention, however, and when contained within the lumens or voids of protective shells, such materials may be considered to be nanoagents for some purposes. Molecular clusters, also known per se, may also be employed as nanoagents in the practice of one or more embodiments of the invention.

The present invention also provides methods for preparing inorganic shells filled with nanoagents as well as methods for their use in biological, scientific and industrial systems. Provision of fullerene nanotubes, especially single- or multi-walled carbon nanotubes having an open end is known. See, e.g. U.S. Pat. No. 6,544,463. Similarly, filling of these nanotubes with fullerenes is also known. It has now been discovered that such nanotubes, especially carbon nanotubes, can be filled with nanoagents having the ability to effect therapeutic, imaging, diagnostic, industrial and scientific utilities to good purpose. It has been found that filling particles or objects tend to collect in the lumens of shells, apparently as a result of lowered free energy through self and niche association in such locations. Accordingly, provision of physicochemical conditions permitting migration of fill materials into such lumens has been found to result in such migration occurring. Conditions amenable for this include especially heating or annealing and dissolving or suspending in a solvent or in materials for population of the lumens themselves. Other conditions which may be useful include sonication, cooling, agitation, stirring or heating at reflux. In some cases, addition of a surfactant facilitates this migration.

It has now also been found that other inorganic shells may be filled with nanoagents in ways similar to those effective for carbon nanotubes. Accordingly, annealing alumina or magnesia hollow shells (having access openings to the lumen) with nanoagent bodies or molecules will result in filling of the lumen with the agents. Similar results can be obtained with solutions and suspensions of nanoagents and these hollow shells.

Some shells, especially ceramic shells, may require etching, abrasion or other treatment to provide physical access to their lumens, although they can typically be synthesized with physical access to their lumens by processes such as anodization. This has been known heretofore. See H. Masuda and K. Fukuda, Science 268, 1466 (1995). When the shells have been filled, they may be coated if desired. Thus, for ceramic shells, such as magnesia, a further coating of ceramic through known deposition techniques may be desired. Coating with organic coating, such as polymer, lipid, or other material may also be preferred, especially in view of the ease of bonding antibodies to such coatings. These coatings may be seen to encase the filler materials—nanoagents within the shells. For some embodiments, this is preferred. It will be appreciated, however, that the filler particles or materials will often remain within the lumen of shells without a coating. In such a case, while the outermost nanoagents may experience the environment external to the shells, those packed within the lumen will not. This is within the spirit of certain embodiments of the invention.

Association of targeting ligand moieties to the surface of inorganic shells may employ any of the several well-known techniques and is not a part of this invention beyond its actual performance as the claims may require. It is often preferred to employ multiple copies of ligand species, or multiple differently targeting ligand species, to effect good targeting to target tissues or loci.

In use, the hybrid materials of the invention comprising filled shells may be used for diagnosis, imaging or treatment in biological systems or in organisms. Biological compatibility is a well understood term and, in general, the shells are biocompatible and employ compatible coatings and the like. The materials are preferably suspended in aqueous medium and administered to a patient or contacted with a tissue or organism. The targeting moieties on the surfaces of the shells localize the shells and their contents to the loci of tissues to which the ligand has been targeted. If the nanoagents filling the shells are radioopaque or lucent, X-ray or other imaging will detect their concentration in such loci. If nanoagents are quantum dots, emissive or other detection techniques locates them. If nanoagents are radioactive or otherwise therapeutically efficacious, then radiation or other therapy occurs at the loci. Many other techniques and protocols may be devised within the spirit of this invention.

Industrial and scientific utility is vast. For example, shells containing imaging capable nanoagents may be employed for imaging biological pollution. By virtue of the high concentration of nanoagents delivered to target loci, subterreanean imaging may be achieved. Similar applications apply to the location of leaks in any fluid transfer system. In these cases, a hybrid with a non-reactive inorganic shell and a functional nanoagent, isolated from the environment, prevents the disruption of either the carrying medium or the nanoagent while providing a clearly imaged detection capability for the migration of the carrying medium away from the fluid transfer system. In addition, the combination of mixtures of hybrids containing different nanoagents, which emit light at different wavelengths or radioactive decay products of different types or at different energies, allow for the marking and tracking of substances or devices with high individual specificity, akin to an individualized, or unique, barcode. Targeted hybrids can be used for the specific location and tracking of biological or chemical species in wet chemical and biochemical assays, such as in chemical sensors and in cell sorting apparati including flow cytometers. Given the size of these hybrids, they can be particularly effective in MEMS or NEMS fluidic devices such as “Labs-on-a-Chip”.

Homeland security and defense utility is similarly vast. For example, shells containing imaging capable nanoagents may be employed in friend/foe identification, for the tracking of strategic materials, components and devices, and for the tagging and tracking of human and material targets.

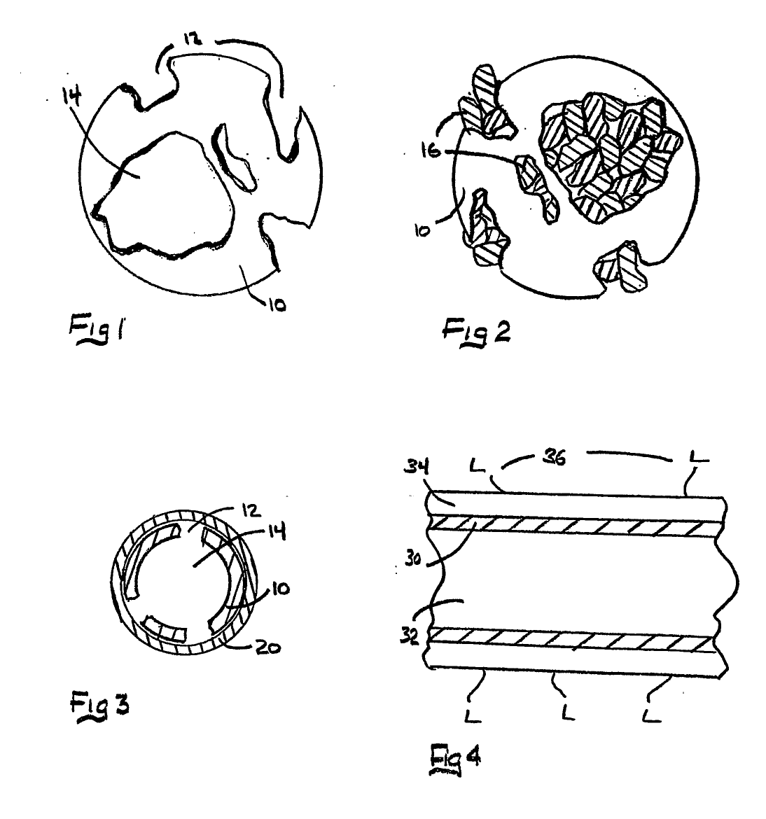

Referring to the drawings, FIG. 1 is a schematic ceramic shell 10 which, in this example, is spherical. It has been etched to provide openings 12 for access to the lumen 14. This shell has been filled in FIG. 2 with nanoagents 16. Some of these protrude from the openings, but generally reside within the lumen.

FIG. 3 shows a cross section of the ceramic spherical shell of FIG. 1. Shell 10 surrounds lumen 14. Openings 12 give access to the lumen from without the shell. Post filling of the lumen with nanoagents, not shown, a coating 20, ceramic, organic or both, may be grown in a conventional way. Ligands, shown as L may be appended to the coating or directly to the shell if desired.

FIG. 4 is of a carbon nanotube. Tube wall, 30 forms the shell with lumen 32. One or more coatings 34 may be appended and ligands, L may be associated with the shell or with a coating on the shell. The nanotube shell may be closed by a coating or by growing carbon end caps.

The hybrid materials of this invention provide a generality in construction. This may be seen as the inorganic shell is pre-existent, or is synthesized prior to filling with the interior species of nanoagent. A broad range of synthesis conditions can be used for the shell, as the synthesis does not require the simultaneous formation of the fill species and the inorganic shell. Also, inorganic shells can be used that are robust over a wide range of synthesis conditions, providing the possibility of synthesizing fill species in the presence of the inorganic shell or within the inorganic shell. A further advantage of the use of a robust inorganic shell is that it can be inert or stable in aggressive or delicate environments. Many interesting, functional nanoparticles, clusters or molecules are toxic to living species. Encapsulating these functional fill species within a bioinert or biocompatible shell that cannot be destroyed by in-vivo processes shields the living species from harm from the fill species. Likewise, the nanoagents are shielded from the biological or other environment.

For example, an inorganic shell composed of primarily of sp2-bonded carbon provides excellent chemical isolation between the living environment and the functional fill species, as there is no process within a living system that can provide sufficient energy to break the carbon-carbon interatomic bonds that form the inorganic shell. Also, many interesting functional nanoparticles, clusters or molecules are unstable in certain chemical environments, which can lead to breakdown and loss of functionality. Containing the functional fill species within a robust, inert inorganic shell allows the functionality of the fill species to be retained in hostile environments.

In some embodiments, it is preferred that nanotubes or cages contain a plurality of boron and/or nitrogen dopant atoms. It is also preferred for some embodiments that the shells comprise unilamellar or multilamellar boron nitride, MoS₂ or WS₂ nanotubes or cages.

A class of hybrid materials in which a variety of fill species are contained within a small set of inorganic shells provides additional important advantages for technological applications. The use of functional nanoparticles, clusters or molecules in a targeted application requires the development of chemical synthesis pathways that provide the bonding of the functional nanoparticles, clusters or molecules with the necessary targeting moiety. In contrast, the use of a small set of inorganic shells allows the chemistry of functionalization of the shell with targeting moieties to be accomplished independent of specific fill species.

An important and unique feature of certain of the hybrid materials of this invention is that a single unit of hybrid material provides the possibility of containing multiple units of the nanoagent fill material within its lumen. This can be a necessary condition for functionality by, for example, improving detection limits (sensitivity) for sensing applications, providing minimum required therapeutic levels, or providing high specific levels of function. For cases where the fill material must be a particular size to retain its function (e.g. quantum dots, dye molecules), the inorganic shell of the hybrid material can sterically prevent the agglomeration or coarsening of multiple fill units while still containing a sufficient total amount of fill material to meet functional requirements.

The inorganic shell of the hybrid material can be of any shape. The exterior size of the inorganic shell may be constrained by particular applications. Particularly interesting shapes are spherical or nearly spherical, oblong or cylindrical. Nanotubes are particular cylindrical inorganic shells that are very useful for hybrid materials. The inorganic shells including nanotubes can be synthesized using one or more of the elements hydrogen, magnesium, boron, carbon, nitrogen, oxygen, calcium, aluminum, silicon, sulfur, titanium, vanadium, molybdenum or tungsten. Particular combinations of these elements are useful, particularly Mg and O; B and N; BN; B, C and N; B and C; C and N; C and Si; Al and O; Ti and C; Ti and N; Ti and O; V and O; S and Mo; S and W. In particular embodiments, the ceramic is MgO, Al₂O₃, TiO₂, Si₃N₄, or SiO₂. In some embodiments, the shells are electrolytically etched, especially etched silica.

For the purposes of this application, the term “nanoagent” is defined to include nanoparticles, quantum dots, organic molecules and molecular clusters (metallic clusters, molecular clusters, semiconducting clusters, semi-metallic clusters, or insulating clusters) and is to be understood to be a functional definition.

The nanoagents provide specific functionality for the system. This functionality may be modified, or dependent upon, the inorganic shell that the fill species is contained within. Of particular interest are nanoparticles or clusters of radioactive isotopes, metallic nanoparticles, magnetic nanoparticles, high mass density nanoparticles, nanoparticles of elements from Group IV, Groups III and V, and Groups II and VI, quantum dots, and individual or clusters of molecules that emit light, transfer charge to, or from, the inorganic shell, or transfer charge through the inorganic shell.

Metallic radionuclides are commonly used as labeling reagents for antibodies in therapeutic and diagnostic applications. For example, radionuclides such as ¹¹C, ¹³N, ¹⁵O, ¹⁸F, ³²P, ⁵¹Mn, ⁵²Fe, ^{52m}Mn, ⁵⁵Co, ⁶²Cu, ⁶⁴Cu, ⁶⁷Cu, ⁶⁷Ga, ⁶⁸Ga, ⁷²As, ⁷⁵Br, ⁷⁶Br, ^{82m}Rb, ⁸³Sr, ⁸⁶Y, ⁸⁹Zr, ⁹⁰Y, ^{94m}Tc, ⁹⁴Tc, ⁹⁵Tc, ^{99m}Tc, ¹¹⁰In, ¹¹¹In, ¹²⁰I, ¹²³I, ¹²⁴I, ¹²⁵I, ¹³¹I, ¹⁵⁴⁻¹⁵⁸Gd, ¹⁷⁷Lu, ¹⁸⁶Re, and ¹⁸⁸Re have been used as labeling reagents for diagnostic imaging techniques. Radionuclides such as ⁵¹Cr, ⁵⁷Co, ⁵⁸Co, ⁵⁹Fe, ⁶⁷Cu, ⁶⁷Ga, ⁷⁵Se, ⁹⁷Ru, ^{99m}Tc, ¹¹¹In, ^{114m}In, ¹²³I, ¹²⁵I, ¹³¹I, ¹⁶⁹Yb, ¹⁹⁷Hg, and ²⁰¹Tl have been used labeling reagents for diagnostic imaging techniques using gamma-ray detection methods. Radionuclides such as ³²P, ³³P, ⁴⁷Sc, ⁵⁹Fe, ⁶²CU, ⁶⁴Cu, ⁶⁷Cu, ⁶⁷Ga, ⁷⁵Se, ⁷⁷As, ⁸⁹Sr, ⁹⁰Y, ⁹⁹Mo, ¹⁰⁵Rh, ¹⁰³Pd, ¹⁵⁹Gd, ¹⁴⁰La, ¹⁶⁹Yb, ¹⁷⁵Yb, ¹⁶⁵Dy ¹⁶⁶Dy, ¹⁰⁵Rh, ¹¹¹Ag, ¹⁹²Ir, ¹⁰⁹Pd, ¹¹¹Ag, ¹¹¹In, ¹²⁵I, ¹³¹_{I,} ¹⁴²Pr, ¹⁴³Pr, ¹⁴⁹ Pm, ¹⁵³Sm, ¹⁶¹Tb, ¹⁶⁶Ho, ¹⁶⁶Dy, ¹⁶⁶Ho, ¹⁶⁹Er, ¹⁷⁷Lu, ¹⁸⁶Re, ¹⁸⁸Re, ¹⁸⁹Re, ¹⁹⁴Ir, ¹⁹⁸Au, ¹⁹⁹Au, ²¹¹At, ²¹¹Pb, ²¹²Bi, ²¹³Bi, ¹³⁷Cs, ⁶⁰Co, ¹⁰⁶Ru, ⁹⁰Sr, ²¹²Pb, ²¹³ Bi, ²²³Ra and ²²⁵Ac has been used in therapeutic applications, such as the targeting of a radiolabeled antibody to a cancer cell. Any radioactive species, such as those listed above, having a reasonable half life in the context of this invention can be employed in the present invention. A nanoparticle of the present can comprise a collection of homogenous or heterogeneous radionuclides. For example a nanoparticle can contain only Re¹⁸⁶ or both Re¹⁸⁶ and Re¹⁸⁸. In a preferred embodiment, radiopharmaceuticals of the present invention comprise isotopes of At, Cs, Co, I, P, Ru, Sr, Cu, As, Rh, Pd, Ir, Ag, Re, Au, Bi, Tc or mixtures thereof. Particularly preferred radioactive isotopes include ²¹¹At, ²¹³Bi, ¹³⁷Cs, ⁶⁰Co, ¹⁹⁸Au, ¹²⁵I, ¹⁹²Ir, ¹⁰³Pd, ³²P, ¹⁰⁶Ru, ⁹⁰Sr, ¹⁸⁶Re, ¹⁸⁸Re, and ^{99m}Tc. Quantum dot materials of particular interest include CdSe, PbSe, CdTe, CdSe/ZnS, CdTe/CdS. Molecular dyes of interest are numerous and are listed in the Handbook of Molecular Probes. See www.probes.com/handbook. Metallic materials that are of interest include Au, Ni, Cu, Ag, Pt, Re, W, 3d transition metals, 4d transition metals, 5d transition metals, Ho, Gd, lanthanide metals, U, actinides, Na, K, alkali metals, Mg, Ca, alkaline earth metals, Al, Ga, and semi-metals. Magnetic nanoparticles include Fe, Co, Ho, Gd, Re, alloys of these elements, and oxides of these elements.

The decay characteristics of radioactive isotopes (half-life, energy and identity of emitted particles (alpha, beta (electron or positron), gamma, neutron, proton) allow the detection of their presence at very low concentrations. In the form of nanoparticles, radioactive isotopes can provide sufficient concentrations to allow statistical confidence for detection or therapeutic efficacy. Radioactive isotopes, or generators that produce radioactive isotopes, can be obtained from the US Dept. of Energy. Persons skilled in, e.g., nuclear medicine appreciate the availability and overall use of such radionuclides. Moreover, nanoparticles containing radioactive isotopes can be synthesized in a wet-chemical lab. For example, certain exemplary technigues are set forth in a U.S. patent application, assigned to the assignee of this application, and entitled Nanoradiopharmaceuticals and Methos of Use, filed on even date herewith as well as U.S. Pat. No. 6,689,338, the disclosures of which are incorporated herein by reference in their entirety and for all

Methods of preparing biocompatible shells whose lumens comprise radioactive nanoparticles may include the steps of providing an inorganic shell, filling the shell with a radionuclide-containing moiety, and reducing the radionuclide-containing moiety inside the shell. In other embodiments, the radionuclide containing moiety will be reduced before insertion into the shell. Radionuclide containing moieties for use in the present invention include nanoparticles comprising compounds having metallic isotopes, which compounds are capable of being reduced by a reducing agent to form a radioactive metal or metal containing composition in nanoparticulate form. The choice of radionuclide for use in the present invention takes into account several of the physical and chemical properties possessed by the radionuclide including the type of radiation emitted by the radionuclide. Radionuclides of the present invention can be alpha, beta, gamma, positron, or electron emitters. The choice of radionuclide also takes into account the energy emission spectrum and the half-life of the radionuclide. For example, the energy emission spectrum and half-life of a radionuclide can be used to calculate the intrinsic radiotherapeutic or radiodiagnostic potency of a radionuclide. A general review of several of the considerations to be taken into account when choosing an appropriate radionuclide can be found in O'Donoghue, J. A. Dosimetric principles of targeted radiotherapy; P. G. Abrams and A. R. Fritzberg (eds.), Radioimmunotherapy of Cancer. New York, N.Y.: Marcel Dekker, 2000; and Goldenberg, J Nucl Med 2002, 43: 693-713, the disclosures of which are incorporated by reference in their entireties. A radioactive nanoparticle can comprise a collection of homogenous or heterogeneous radionuclides

A reducing agent is a compound that reacts with a moiety in a relatively oxidized form, for example, a metallic radionuclide in a relatively high oxidation state. The reducing agent acts to lower its oxidation state by transferring electron(s) to the radionuclide. The resulting, reduced material, preferably a metal oxide, where the metal contains radioactive isotopic species, can attain the form of nanoparticles with controlled mean diameters. Suitable reducing agents are those that are capable of quickly reducing a radionuclide moiety in accordance with the present invention. Suitable reducing agents for the synthesis of the radioactive nanoparticles of the present invention include, but are not limited to, stannous salts, dithionite or bisulfite salts, borohydride salts, and formamidinesulfinic acid, wherein the salts are of any pharmaceutically acceptable form. Metal hydrides, especially borohydrides such as sodium borohydride are preferred. By controlling the rate of reduction of the radionuclide, the size of the nanoparticles can be controlled. Faster reduction rates result in smaller particles. In one aspect, the reduction rate can be controlled by hydrogen ion concentration, e.g., pH. The amount of a reducing agent used will depend upon the amount of radionuclide to be reduced and can be determined by a skilled practitioner. Reducing agents are chosen dependent on the radionuclide to be reduced. For example, for reduction of rhenium isotopes, a preferably reducing agent is a metal hydride, e.g., borohydride. The radioactive nanoparticles can be in compositions further comprising one or more ligand, such as a stabilizing ligand or performance enhancing material. Exemplary among these are polymers which keep the particles in effective suspension or which interfere with agglomeration or other undesired association.

Radioimmunotherapy (RIT) involves the use of antibodies or other biologically active ligands to deliver radionuclides to cells bearing the corresponding antigen. It has proved useful in the treatment of diffuse or occult malignancies that cannot be successfully managed by surgical excision or other localized approaches. A limitation of prior forms of targeted radiotherapy is that the dose rate per cell is lower than can be achieved by conventional brachytherapy (seed implantation). In the present embodiment, nanoparticles containing from 1% to 95% radioactive isotopes are used to deliver sufficient therapeutic doses at higher dose rates.

For this and other embodiments, targeting ligands can be synthetic, semi-synthetic, or naturally-occurring. Exemplary targeting ligands for use in the present invention include, but are not limited to proteins, including antibodies, glycoproteins and lectins, peptides, polypeptides, saccharides, including mono-and polysaccharides, vitamins, steroids, steroid analogs, hormones, cofactors, bioactive agents, and genetic material, including nucleosides, nucleotides and polynucleotides.

In some embodiments, the targeting agents specifically target receptors on or near selected biological targets. The term “receptor” as used herein refers to a molecular structure within a cell or on the surface of the cell which is generally characterized by the selective binding of a specific substance. Exemplary receptors include, for example, cell-surface receptors for peptide hormones, neurotransmitters, antigens, complement fragments, and immunoglobulins, cytoplasmic receptors for steroid hormones and receptors on invading pathogens. Receptors can be, for example, membrane bound, cytosolic or nuclear; monomeric (e.g., thyroid stimulating hormone receptor, beta-adrenergic receptor) or multimeric (e.g., PDGF receptor, growth hormone receptor, IL-3 receptor, GM-CSF receptor, G-CSF receptor, erythropoietin receptor and IL-6 receptor). In some embodiments, the targeting agents specifically target proteins on or near selected biological targets

The nanoparticles may be chemically pure, e.g. a mixture of radioactive and non-radioactive isotopes of the same chemical element. The nanoparticles may also be of a chemical compound that contains the radioactive isotope (oxide, nitride, etc.). The nanoparticles may contain a segregated mixture of different chemical forms, e.g. a core pure chemical with a surface oxide layer and each of these forms can be useful. With certain isotopes, compound formation may be a non-deleterious event that occurs during the synthesis of the nanoparticles and not impede utility, e.g. formation of rhenium oxide during the synthesis of ¹⁸⁶Re or ¹⁸⁸Re containing nanoparticles.

Radioactive Tagants may be employed. The isotope-specific decay characteristics of radioactive isotopes can be used as an effective means to provide tracking of materials, devices or objects. Compared to detection of chemicals and biological species, the detection of radioactive elements can be achieved with simple equipment and with stealth. Levels of radioactive decay necessary for detection can be much lower than levels of radioactive decay that is harmful to biological species including humans. Thus, such tagants can facilitate the monitoring of biological organisms, tissues or individuals. Such tagants may also keep track of non-biological things such as items of inventory, goods in transit or in manufacturing, dangerous or strategic materials, such as weapon components, nuclear material, explosives and the like. When associated with a targeting moiety, or with a construct that naturally accumulates in specific organs, radioactive isotopes can be effective sensors for biomedical applications and for tracking individuals.

As the nanoagents are contained within an inert, inorganic container, these elements provide the means to detect the presence of controlled chemicals or dual use chemicals at suspect weapons of mass destruction production or storage sites, while not affecting legal industrial production and use or agricultural use of the chemicals. The ability to control the quantity of the isotope(s) used as a marker and to choose isotopes with specific half-lives provide the capability to build an effective “expiration date” into the marker by controlling the time until the radioactive levels drop to negligible levels or drop to below a threshold level.

The combination of two or more radioactive isotopes in a nanoparticle, and/or the combination of two or more nanoparticles, each containing a single radioactive isotope specie, within a single shell can provide a large number of uniquely identifiable markers. Control of the relative proportions of isotopes within the nanoparticles, and/or control of the size and number of nanoparticles within a unit provides additional factorial increases in the number of specific and unique radioactive decay markers. The use of two or more unique hybrid units in known proportions can also be used as a means to provide unique radioactive decay markers. The ability to combine different isotopes in different proportions provides an essentially unlimited number of uniquely identifiable markers and can achieve a radioactive decay “barcode.

Magnetic nanoparticle nanoagents may be employed in some embodiments of this invention. While paramagnetism can be present at very small dimensions, ferromagnetism requires a minimum size in three dimensions. Nanoparticles therefore provide a construct that can provide ferromagnetic behavior as well as strong magnetic response and detectability for spin resonance-based techniques such as electron spin resonanace (ESR) and nuclear magnetic resonance (NMR). Useful elements for this include Fe, Co, Ho, and Gd. Useful elements for magnetic nanoparticles include iron, cobalt, chromium, dysprosium, erbium, europium, gadolinium, nickel, manganese, holmium, terbium, thulium, vanadium, neodymium and alloys of these elements. Chemical compounds of these elements, such as bromides, carbonates, chlorides, fluorides, iodides, nitrates, oxides, phosphates, sulfates or sulfides, can also be useful. Magnetic nanoparticles can be synthesized in a wet chemical lab. Certain magnetic nanoparticles can also be purchased from companies such as Reade Advanced Materials (http://www.reade.com) and, in any event, are known per se. Hybrids containing magnetic nanoparticles containing elements that can also be used in magnetic resonance imaging (MRI) can enhance the signal and detectability of diagnostic techniques employing such agents. Hybrid materials containing magnetic nanoparticles can be used as active components in devices including MEMS, NEMS, fluidic, electro-optic, electronic and other systems.

One embodiment of a hybrid material containing magnetic nanoparticles is for MRI. In this embodiment, a strong NMR signal from the hybrid is produced by containing one or more magnetic nanoparticles within an inorganic shell. The inorganic shell is functionalized with a targeting moiety or naturally accumulates, e.g. in a specific organ. In another embodiment, a hybrid composed of an inorganic shell containing magnetic nanoparticles is used as an actuator in a device. In this case, application of a magnetic field to the actuator induces switching of the hybrid from one state to another. In this embodiment this switching could be as a valve controlling flow in a fluidic device such as a lab on a chip, or as a structural component that changes the orientation of an optical component, or as a means to modify the resonance of a filter component of an electro-optic or electronic device.

High mass density nanoparticles have many uses in the context of this invention. High mass density nanoparticles within an inorganic shell can provide a useful means to design in specific mechanical properties, such as a particular resonance frequency, switching latency due to inertial response, Q factor, frequency selectivity of a resonator. Useful elements for high mass density nanoparticles are the elements of Periods 5, 6 and 7 of the Periodic Table of the Elements including the Lanthanides and Actinides. In general, the Inert Gases will not be useful for this purpose. For some applications, especially in which the inorganic shell is composed of light elements, the elements in Period 4 of the Periodic Table of the Elements can be useful. In general, high mass density nanoparticles are nanoparticles in which the mass density exceeds the mass density of the inorganic shell by a factor of five or more. For this calculation, the volume of the empty lumen of the inorganic shell is included in the density calculation of the inorganic shell.

High mass density nanoparticles can be synthesized in a wet chemical lab through known techniques. One embodiment comprises a nanotube filled, or partially filled, with high mass density nanoparticles. This nanotube is integrated within a device as a resonator and is used to frequency select particular electromagnetic signals pertinent to the performance of the device.

Quantum Dot (QD) is the term given to small particles—nanometer to micron—in which confinement of the electrons due to the size of the particle yields quantum effects and the production of a modified electronic band structure with specific energy levels. This modified electronic structure can be useful for a number of applications. For the purposes of this document, we refer to QDs in which strong and efficient light emission can be produced. Since the frequency of emitted light is a function of the energetic separation of the discrete energy levels within the QD, and these energy levels are determined by the quantum confinement of the electrons of the material resulting from the particles size, the size of the QD determines the wavelength of light emitted for each particular element or compound. It is therefore usually importantl that the QDs not change size for stable functioning of a device. The most common path by which QDs can change size is through agglomeration in which size increases; another path is through coarsening. Chemical attack on the QD could reduce the size of the QD. It is, thus, to be avoided as is the case when QDs are encased in the shells of this invention.

Specific QDs of interest for some embodiments include CdSe, PbSe, CdTe, ZnS coated QDs, CdS coated QDs, CdSe/ZnS, CdTe/CdS. Quantum dots can be obtained from Evident Technologies, Inc. (http://www.evidenttech.com) and other sources known to the art. Metallic and Semiconducting materials will form QDs when they are in nanoparticle form. A number of metallic nanoparticles have been made that have optical properties that differ from the bulk material, including Au, Ag and Pt. Nanoparticles of these materials can be synthesized using wet chemical methods, are generally known and methodologies can be found on the world wide web.

Metallic and/or semiconducting nanoparticles can be nanoagents or included with nanoagents as contemplated by certain embodiments hereof and may be loaded into inorganic shells to form hybrid materials, Moreover, blended nanoparticles may provide certain advantages over single types of nanoparticle alone. Thus, this invention also comprehends hybrid materials, in which nanoparticles known to possess useful optical properties are combined within a single hybrid material, or mixtures of hybrid materials, with each hybrid unit containing a specific nanoagent type.

Quantum dots, which may comprise the hybrid materials of this invention can be bright optical emitters that emit over a narrow frequency range and can, therefore, be used as a tag to indicate the presence of the specific hybrid material in which it is contained. A further function of QDs contained within hybrid materials can be as an element in a quantum device, such as a memory element, or as a component of a q-bit of a quantum computer, or as a component of an individual electronic logic circuit. Hybrid material containing QDs can be used to identify specific targets by attaching to the target with the QD providing a light signature for the presence of that target. One embodiment of this is the use of Inorganic shell/QD hybrids as a means to identify specific cells, such as cancer cells, within a cell sorting and identification system as used in flow cytometry. In this embodiment, an inorganic shell containing a targeting moiety, such as an antibody for a specific antigen on a cancer cell, contains a QD that emits light of a particular wavelength. The presence of this cancer cell is than identified by the detection of this light.

It is also useful to employ two or more hybrids or nanoagent containing shell types, each with different targeting moieties, each with specific affinity for different antigens on a cell membrane, and each containing different QDs that emit light at different wavelengths. The presence of both wavelengths of light provides surety of a positive identification of a target cell. In another embodiment, different targeting moieties for the target cancer cell can be attached to every hybrid shell, again providing greater surety in the positive identification of the target cell. A further embodiment is to use inorganic shell/QD hybrids for applications in which higher light intensity than can be obtained from a single QD is necessary. In this embodiment, the inorganic shell would be loaded with two or more QDs to provide greater intensity. A further advantage of this embodiment is that the specific geometry of the inorganic shell can enable the loading of multiple QDs while preventing or greatly mitigating against, the agglomeration or coarsening of the QDs, an event that is deleterious to the function of QDs and renders them useless as an indicator.

Individual molecules, or clusters of molecules that emit light can also be used as nanoagents in hybrids of this invention. These cluster molecules emit light at well-defined wavelengths due to their molecular orbital structure, which contains discrete energy levels. One class of these molecules are known as dye molecules. Dye molecules can be purchased from general chemical suppliers such as Aldrich Chemical of Milwaukee, Wis. Dye molecules can also be synthesized using well known synthetic organic chemistry methods.

One function of light-emitting molecules contained within the lumens of inorganic shells is as a bright optical emitter that emits over a narrow frequency range and can therefore be used as a tag to indicate the presence of the specific hybrid material in which it is contained. The hybrid material containing the light-emitting molecules can be used to identify specific targets by attaching to the target with the light-emitting molecules providing a light signature for the presence of that target. One embodiment of this is in the use of Inorganic shell/light-emitting molecule hybrids as a means to identify specific cells, such as cancer cells, within a cell sorting and identification system as used in a flow cytometer. In this embodiment, an inorganic shell containing a targeting moiety, such as an antibody for a specific antigen on a cancer cell, contains light-emitting molecules that emit light of a particular wavelength. The presence of this cancer cell is than identified by the detection of this light.

A further refinement of this embodiment includes the use of two or more hybrids, each with different targeting moieties, each with specific affinity for different antigens on a cell membrane, and each containing light-emitting molecules that emit at a different wavelength. The presence of both wavelengths of light provides higher surety of a positive identification of a target cell than one species alone. A further embodiment uses inorganic shell/light-emitting molecule hybrid materials for applications in which higher light intensity than can be obtained from a single light-emitting molecule is necessary. In this embodiment, the inorganic shell would be loaded with multiple copies of the light-emitting molecule to provide greater intensity.

The nanoagents may be filled into inorganic shells by several methods. In one method, the fill material is brought into contact with the inorganic shell that has been prepared to be ready for filling by etching or otherwise or is naturally open. The fill material than fills the inorganic shell through self-assembly, or by being induced to enter. This can be accomplished through a variety of routes including vapor, liquid and solution/suspension routes. The driving force for filling of the inorganic shell will depend on the specific combination of inorganic shell and fill species. In most cases, entering the lumen of an inorganic produces a higher coordination between the fill species and the inorganic shell than on the exterior of the shell. This improved coordination yields an improvement of the energetics of interaction between the fill specie and the inorganic shell thereby driving the filling. Except in unusual circumstances involving strong charge transfer materials, such as alkali metals, this improved coordination is the dominant effect in the filling process. Other drivers for filling can be electronic, or structural (steric). Steric effects result when a fill material can achieve a superior (lower energy) conformation inside the inorganic shell than exterior to the inorganic shell.

Another method for filling is from the vapor phase. In circumstances in which a vapor of a fill material can be produced, exposing the vapor to open inorganic shells is an efficient method to fill. Most nanoparticles and clusters will not be amenable to this method as their vapor pressures are extremely low. However, some fill materials, especially molecules that emit light are amenable to this procedure.

In another method, precursors to the fill material are brought into contact with the inorganic shell that has been prepared to be ready for filling or is naturally open. This can be accomplished through a variety of routes including vapor, liquid and solution/suspension routes. The precursors to the fill material than fill the inorganic shell through self-assembly, or by being induced to enter. After the precursors are contained in the inorganic shell, the precursors are reacted together to produce the fill material, or undergo some reaction to yield the fill material. The fill material, or precursor material are brought into contact with the open inorganic shell in the form of a liquid (melt), vapor or solution/suspension. A common way to induce the liquid or vapor state is by annealing the inorganic shells in the presence of a fill molecule.

Inorganic shell useful in the present invention have lumens. The lumens will contain nanoparticles, material clusters, or certain types of molecules with specific function—collectively, nanoagents. The overall shape of the hybrid materials will be dictated by the shape of the inorganic shell. Inorganic shells that are preferred are robust over a wide range of synthesis conditions providing the possibility of synthesizing fill species in the presence of the inorganic shell or within the inorganic shell. A second advantage of the use of a robust inorganic shell is that it can be inert or stable in aggressive or delicate environments. For example, an inorganic shell composed of sp2-bonded carbon provides excellent chemical isolation between the living environment and the functional fill species, as there is no process within a living system that can provide sufficient energy to break the carbon-carbon interatomic bonds that form the inorganic shell. Containing the functional fill species within a robust, inert inorganic shell allows the functionality of the fill species to be retained in hostile environments.

The use of a small set of inorganic shells allows the chemistry of functionalization of the shell with targeting moieties to be accomplished independent of specific fill species. Moreover, the size of the inorganic shell can be tailored to the fill species and to the desired use. The inorganic shell of the hybrid material can be of any shape. The exterior size of the inorganic shell may be constrained by particular applications. Particularly interesting shapes are spherical or nearly spherical, oblong or cylindrical. Nanotubes are particular cylindrical inorganic shells that are very useful for hybrid materials. The inorganic shells including nanotubes can be synthesized using one or more of the elements hydrogen, magnesium, boron, carbon, nitrogen, oxygen, calcium, aluminum, silicon, sulfur, titanium, vanadium, molybdenum or tungsten. Particular combinations of these elements are useful, particularly Mg and O; B and N; BN; B, C and N; B and C; C and N; C and Si; Al and O; Ti and C; Ti and N; Ti and O; V and O; S and Mo; S and W.

Production of hybrid materials of this invention requires one or several steps. A first processing step usually involves producing an opening in an existing inorganic shell. This step is not required in applications in which the inorganic shell already contains an opening of sufficient size to allow the fill species to enter. An example of this case is with anodized, electrochemically-etched or chemically etched oxide nanomaterials made of alumina, magnesia, a titanium oxide, etc. When necessary to open the inorganic shell, this can be accomplished by heating in an oxidative atmosphere in one embodiment, heating in a reductive atmosphere in another embodiment, or through chemical treatment with acids or bases in further embodiments. Effective heating temperatures have been found, depending on the specific material systems, to be in the range of 200 C to 1000 C. Particular oxidizing atmospheres that show utility are wet air, oxygen, carbon dioxide, an argon/oxygen mixture, an inert gas/oxygen mixture, or steam. Particular reducing atmospheres that show utility are hydrogen, carbon monoxide or reducing organic gases. Chemical treatments with acids that show utility use nitric acid, phosphoric acid, sulfuric acid, triflic acid, chlorosulfonic acid, hydrochloric acid, hydrofluoric acid or a super acid. Chemical treatments with bases that show utility use NaOH or KOH. In some cases, it si effective to conduct chemical treatment under refluxing conditions. It can be helpful, especially in order to control kinetics of reactions, to use acids or bases that have been diluted with water.

Prior to the opening step, or sometimes after, it is necessary to conduct a cleaning of the inorganic shells prior to filling. This can be accomplished through heating in an inert atmosphere, in an oxidizing atmosphere, or in vacuum, or by treating with various organic solvents in through wet chemical processing. Heating in inert atmospheres of hydrogen, nitrogen, helium or argon at temperatures above 100 C and at, or below, 800 C is useful. Heating in vacuum at pressure levels between 10⁻³ torr and 10⁻¹¹ torr, in temperatures between 200 C and 1600 C, and over times from seconds (flash heating) to 62.5 hours, depending on inorganic material and impurity, shows utility. Particular oxidizing atmosphere that show utility are wet air, oxygen, carbon dioxide, an argon/oxygen mixture, an inert gas/oxygen mixture, or steam at temperatures, depending on material, at temperatures from room temperature to 400 C.

A further desired processing step is to seal the inorganic shell. This step is not always necessary; In many cases, especially when the inorganic shell is structurally commensurate with the fill specie, the strong energetic driving force that yields filling effectively prevents the motion of the fill species back out of the inorganic shell. Also, the fill specie can act to be the sealant of the inorganic shell as suggested in FIG. 2. In situations where this is necessary or desired, a variety of chemical reactions can be used to seal the entrance of the inorganic shell. This process can include the functionalization of the edges of the shell opening with a chemically-connected ligand such as carboxylic acid. Another approach provides for the regrowth of the inorganic shell through the chemical placement of precursor compounds at the site of the shell opening through chemical attachment followed by chemical reactions to form the shell. These chemical reactions could include pyrolysis, oxidation, reduction, or hydrolysis and could be induced by heating in vacuum or particular gases, electron, ion or neutron irradiation, exposure to visible, infrared, or ultraviolet light, exposure to x-rays, titration, treatment with acids, bases, or organic compounds. Examples of this are the placement of boron-containing materials such as borazine and reaction in a N₂ atmosphere to regrow boron nitride shells, pyrolysis of carbon containing compounds such as amorphous carbon, polymers, and hydrocarbons for the regrowth of carbon shells.

The compositions of this invention provide a general architecture at the sub-micron scale that can be used in a number of applications. In some embodiments, the overall size of the hybrid material could exceed one micron in one or more dimension. In one embodiment, the hybrid material is used as a targeted therapeutic for the treatment of specific classes of metastatic cancers involving solid tumors in which the provision of a minimum level of localized radiation dose and dose rate is necessary to achieve therapeutic efficacy. The use of the inorganic shell removes the possibility of deleterious chemical interactions within the body. A second application embodiment is through the use of optical emitters such as quantum dots or molecular dye molecules or clusters within the inorganic shell to provide light emission for sensing or diagnostic applications. In general this embodiment involves the presence of specific targeting moieties on the exterior of the inorganic shell These applications can also give rise to chemical sensors.

In one preferred embodiment, a novel system couples the advantages of conventional radioimmunotherapy and liposomal drug delivery systems. An inorganic shell is provided whose lumen is filled with radionuclides. Certain inorganic shells, and specifically unilamellar and multilamellar carbon nanotubes and graphitic cages, have the advantages of a large interior volume, impermeability to atomic species, and the ability to be filled with various inorganic compounds under even harsh processing conditions and are preferred for some applications. A phospholipid coating on the outside of the shell. This coating can be a monolayer if the shell is hydrophobic, or a bilayer if the shell is hydrophillic. The lipids can be modified with PEG to tailor the circulation time of the construct. They can also be modified with fluorescent labels or other markers to be used for supplemental tracking.

Antibodies may or may not be attached to the construct, depending on whether the radiotherapy is to be directed to a specific target cell by way of antigen binding. If used, the antibodies are selected and attached in conventional ways. One embodiment of this disclosure is illustrated in FIG. 4 where a carbon nanotube is filled with nanoagents comprising radionuclides. The nanotube is coated and ligand applied. Such constructs enable the treatment of both systemic disease (e.g. leukemia) and localized, vascular tumors (e.g. prostate cancer) that are traditionally managed by more invasive procedures. Localization by both immunochemistry and by the ‘leaky endothelium’ model is possible. In principle, the construct is also compatible with a host of radionuclides, which is important because different radionuclides yield different success rates in the treatment of certain malignancies. Unlike some conventional radioimmunotherapeutic agents, the construct can be designed so that it is too large to cross the blood-brain barrier, reducing the risk that radionuclides will be delivered to unintended locations in the body. The lumen of an inorganic shell can be large, capable of encapsulating hundreds or thousands of individual atoms, so it may be possible to achieve higher dose rates and total doses than with other forms of RIT. Finally, promising shell materials like carbon nanotubes can be produced with high efficiencies, in contrast to certain alternatives for molecule-based radiotherapy like metallofullerenes.

Existing inorganic shells are made porous by sonication or reflux in a strongly oxidizing acid (e.g. HNO3/H2SO4). The open shells are then recovered by sedimentation or filtration. These are then resuspended in a solution containing the metal salt of the fill species, e.g. M+. The M+ ions permeate into the shells' lumens due to diffusion down the concentration gradient until an equilibrium is reached. The shells containing M+ ions are then recovered with a desalting column into water or some buffer deficient in species that can be easily reduced (including M+).

At this point a very large excess of reducing agent is added. Because the concentration gradient for the reducing agent (entering the shell) is greater than the concentation gradient for M+(leaving the shell), reducing agent will diffuse in more rapidly than M+will reduce out. The net result is the reduction of M+ to M inside the lumen of the shell. Once the concentration of the reduced species inside a shell exceeds the critical value, a nanoparticle of M is nucleated. This nanoparticle is too large to permeate the walls of that shell and therefore is sterically trapped inside the lumen. The filled shells can be recovered as above.

The present specification set forth certain preferred embodiments of the invention. Other aspects thereof will be apparent to persons of skill in the art and all such are comprehended hereby.

1. A composition comprising a plurality of inorganic, shells having lumens, at least one dimension of said shell being between about 5 and about 500 nanometers, the lumens containing a plurality of nanoagents.

2. The composition of claim 1 wherein at least some of the shells are associated with targeting ligand.

3. The composition of claim 1 wherein at least some of the shells are coated with a biocompatible coating.

4. The compound of claim 3 wherein the coating is a monolayer or bilayer phospholipid.

5. The composition of claim 3 wherein the coating is a polymer.

6. The composition of claim 3 wherein the coating is a polyalkylene polyol.

7. The composition of claim 3 wherein the coating is associated with targeting ligand.

8. The composition of claim 7 wherein the targeting ligand is specific for a biological target.

9. The composition of claim 7 wherein the ligand is an antibody.

10. The composition of claim 7 wherein the ligand is a protein.

11. The composition of claim 1 wherein the nanoagents are substantially isolated from the environment external to the shells.

12. The composition of claim 1 wherein the shells comprise ceramic.

13. The composition of claim 12 wherein the ceramic is MgO, Al₂O₃, TiO₂, Si₃N₄ or SiO₂.

14. The composition of claim 1 wherein the shells are electrolytically etched.

15. The composition of claim 1 wherein the shells comprise SP² bonded carbon.

16. The composition of claim 13 wherein the shells are unilamellar or multilamellar carbon nanotubes or graphitic cages.

17. The composition of claim 16 wherein the nanotubes or cages contain a plurality of boron and/or nitrogen dopant atoms.

18. The composition of claim 1 wherein the shells comprise unilamellar or multilamellar boron nitride, MoS₂ or WS₂ nanotubes or cages.

19. The composition of claim 1 wherein at least some of the shells are associated with fluorescent or phosphorescent label.

20. The composition of claim 1 suspended in a biologically compatible fluid.

21. The composition of claim 1 wherein the nanoagents comprise radionuclides.

22. The composition of claim 21 wherein the radionuclides are ²¹¹At, ²¹³Bi, ¹³⁷ Cs, ⁶⁰Co, ¹⁹⁸Au, ¹²⁵I, ¹⁹²Ir, ¹⁰³Pd, ³²P ¹⁰⁶Ru, ⁹⁰Sr, ¹⁸⁶Re, ¹⁸⁸Re, or ⁹⁹Tc.

23. The composition of claim 1 wherein the nanoagents comprise quantum dots or optically active nanoparticles.

24. The composition of claim 23 wherein the quantum dots are comprised of CdSe, PbSe, CdTe, ZnS coated Quantum Dots, CdS coated Quantum Dots, CdSe/ZnS, or CdTe/CdS.

25. The composition of claim 1 wherein the nanoagents comprise organic marker, radiodense material, reporter molecule or dyes.

26. The composition of claim 25 wherein the reporter molecule or dyes are molecules that fluoresce at a defined wavelength when excited with higher energy light.

27. The composition of claim 25 wherein the reporter molecule or dyes have a high quantum yield.

28. The composition of claim 1 wherein the nanoagents comprise molecular clusters.

29. The composition of claim 1 wherein the nanoagents comprise magnetic nanoparticles.

30. The composition of claim 29 wherein the magnetic nanoparticles comprise iron, cobalt, chromium, dysprosium, erbium, europium, gadolinium, nickel, manganese, holmium, terbium, thulium, vanadium, neodymium, or alloys thereof.

31. The composition of claim 30 wherein the nanoparticles comprise one or more bromide, carbonate, chloride, fluoride, iodide, nitrate, oxide, phosphate, sulfate or sulfide.

32. A method of preparing inorganic shells filled with nanoagent comprising:
providing biologically compatible, inorganic shells having at least one dimension between about 30 and about 500 nanometers and having lumens with access external to the shells; and
placing the shells and the nanoagent intended for filling the shells into a physicochemical environment effective for inducing the nanoagent to enter the lumens.

33-70. (canceled)

71. A method of imaging a biological system comprising introducing into the system a composition comprising a plurality of inorganic shells having lumens, at least one dimension of said shell being between about 30 and about 500 nanometers, the lumens containing a plurality of nanoagents.

72-77. (canceled)

78. A method of treating a disease state in a biological organism comprising contacting the organism with a composition comprising a plurality of inorganic, biocompatible shells having lumens, at least one dimension of said shell being between about 30 and about 500 nanometers, the lumens containing a plurality of nanoagents; said nanoagents being therapeutically efficacious.

79-82. (canceled)
